(19) United States
(12) Patent Application Publication
(10) Pub. No.: US 2008/0033321 A1
(43) Pub. Date: Feb. 7, 2008

(54) Device and Method for Detecting Plagiocephaly in Infants

(75) Inventors: Ricardo Hahn, Sierra Madre, CA (US); Raymond Peck, Los Angeles, CA (US)

Correspondence Address: MCDERMOTT WILL & EMERY LLP, Suite 3400, 2049 Century Park East, Los Angeles, CA, 90067, US

(73) Assignee: Alfred E. Mann Institute for Biomedical Engineering at the Univ. of Southern California

(21) Appl. No.: 11/672,153

(22) Filed: Feb. 7, 2007

Related U.S. Application Data
(60) Provisional application No. 60/821,355, filed on Aug. 3, 2006.

(51) Int. Cl. A61B 5/107

(52) U.S. Cl. 600/587

(57) ABSTRACT

Disclosed are devices and methods for detecting plagiocephaly in infants. The one-piece, semi-rigid device is positioned on the infant by placing the caliper's ear-tips in the infant's ears and positioning a planar connector segment approximately under the nose. A physician or caregiver can visually detect plagiocephaly by detecting misalignment between a center marker on the planar segment and the midline of the infant's nose.

### CROSS REFERENCE TO RELATED APPLICATIONS

This application is related to and claims the benefit of the filing date of U.S. Provisional Application Ser. No. 60/821,355, entitled “Caliper and Method for Detecting Plagiocephaly in Infants,” filed Aug. 3, 2006, attorney docket no. 64693-171, the contents of which are incorporated herein by reference.

### BACKGROUND

1. Field

The application relates to a device for detecting plagiocephaly in infants.

2. General Background

Medical studies have shown that newborn infants who sleep on their backs have reduced risk of dying suddenly from Sudden Infant Death Syndrome (SIDS), compared to infants who sleep on their stomachs. Efforts by the American Academy of Pediatrics to disseminate this information to pediatricians and parents have resulted in an increase in the practice of placing infants in a supine position for sleeping.

An unanticipated effect of the increasingly widespread practice of placing infants in a supine position for sleeping has been an increase in the number of infants developing positional plagiocephaly, i.e. a flat or misshapen area on the back of the skull. A newborn infant's skull is relatively deformable due to flexibility of the bone plates and non-fusion of the sutures between adjacent bone plates. This property of deformability permits the infant's head to pass between the bones of the mother's pelvis during birth. As the infant matures, however, the bone plates of the skull become increasingly rigid and the sutures eventually fuse.

When an infant spends many hours sleeping or resting exclusively on its back, the bones at the back of the skull tend to flatten from pressure against the sleeping surface due to the weight of the infant's head. If the practice of sleeping exclusively on the back is continued through the critical period during which the bones of the skull become rigid, the flat or misshapen area on the skull can become permanent.

Parents often do not detect plagiocephaly; rather physicians are the first to detect plagiocephaly. Sometimes plagiocephaly is later detected using conventional measuring devices that measure the size, dimensions, and shape of the infant's cranium.

Measuring head or cranial shape has been a mainstay in the treatment of cranial synostosis for decades, but with the increasing prevalence of positional plagiocephaly it has become a major reason for referral to neurosurgical clinics by pediatricians and by parents. Conditions of the head such as sagittal synostosis resulting in a long and narrow head, can be suspected or even diagnosed on the basis of cranial measurements, as can the flattening of the back or asymmetrical flattening of the head related to the position in which a baby sleeps. Orthotic devices are often used to treat conditions which do not actually require surgery, and the ability to make inexpensive and repeated measurements would be useful in evaluating the need for treatment of a particular patient as well as the response to treatment of any individual patient, and the effectiveness of any particular therapeutic intervention.

Traditionally, measurements of cranial shape have been made either on radiographs which require radiation exposure, or with the use of large calipers which require the points of the calipers to be placed at particular locations on an infant's head. This introduces significant operator dependency on the accuracy of the measurement, and cost and complexity of the calipers has limited their use generally to highly-skilled neurosurgeons and others who see a large number of these cases.

### SUMMARY

A plagiocephaly detection device and related method allow physicians and caregivers to detect plagiocephaly on newborn infants.

In one aspect of the plagiocephaly detection device and method; a plagiocephaly detection device comprises a one piece structure having curved calipers attached at their distal ends by a substantially planar connector bridging the calipers. Each caliper further comprises an ear tip at the proximal end. The ear tips are angled toward the inner area of the device. The curved calipers of the device complement the round contours of the infant's face and provide flexibility and comfort. The planar connector comprises spatial markings which fall under the infant's nose while the ear tips are positioned in the infant's ears.

The device may have a plurality of sizes and dimensions to be used on newborns of 6 months or younger. In some embodiments, the device may provide visual indications of asymmetry, rather than quantitative measurements.

It is understood that other embodiments of the plagiocephaly detection device and methods of use will become readily apparent to those skilled in the art from the following detailed description, wherein it is shown and described only exemplary embodiments by way of illustration. As will be realized, the plagiocephaly detection device and methods are capable of other and different embodiments and its several details are capable of modification in various other respects. Accordingly, the drawings and detailed description are to be regarded as illustrative in nature and not as restrictive.

### BRIEF DESCRIPTION OF THE DRAWINGS

Aspects of the device and method are illustrated by way of example, and not by way of limitation, in the accompanying drawings, wherein:

FIGS. 1A-B illustrate a top view of a shape of a normal skull and a skull exhibiting plagiocephaly.

FIG. 2 is a side view diagram of the plagiocephaly detection device.

FIG. 3 is a front view of plagiocephaly detection device.

FIG. 4 is an oblique view of plagiocephaly detection device.

FIG. 5 is a front view of an exemplary embodiment of the plagiocephaly device in use.

FIG. 6 is a top view of an exemplary plagiocephaly detection device in use.

FIG. 7 is a top view of an exemplary plagiocephaly detection in use.

### DETAILED DESCRIPTION

The detailed description set forth below is intended as a description of exemplary embodiments of the plagiocephaly detection device and methods and is not intended to represent the only embodiments in which plagiocephaly detection device and methods can be practiced. The term “exemplary” used throughout this description means “serving as an example, instance, or illustration,” and should not necessarily be construed as preferred or advantageous over other embodiments. The detailed description includes specific details for the purpose of providing a thorough understanding of the plagiocephaly detection device and methods. However, it will be apparent to those skilled in the art that the plagiocephaly detection device and methods may be practiced without these specific details. In some instances, well-known structures and devices are shown in block diagram form in order to avoid obscuring the concepts of the plagiocephaly detection device and methods.

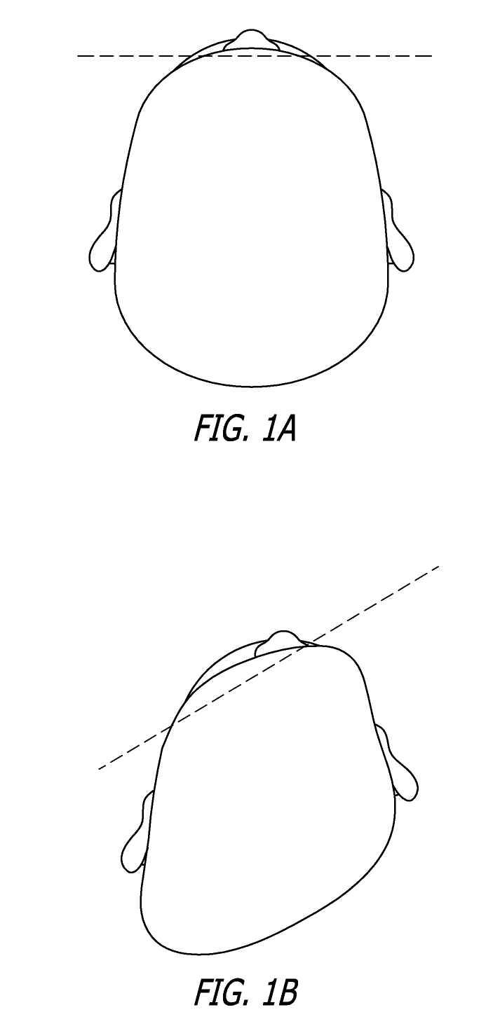

FIGS. 1A-B illustrate a top view of a normal to plagiocephaly skull. FIG. 1 depicts a normal skull with correct symmetry of skull, nose and ears. As is seen in FIG. 1B, one of the manifestations of plagiocephaly is asymmetry developed between the ears and the nose of the skull, which results from cranial deformation. The right side of the skull, along the right ear, is shifted forward from its correct axis or its natural position as shown in FIG. 1A of a normal skull. In reality both ears should be of a similar distance from the nose.

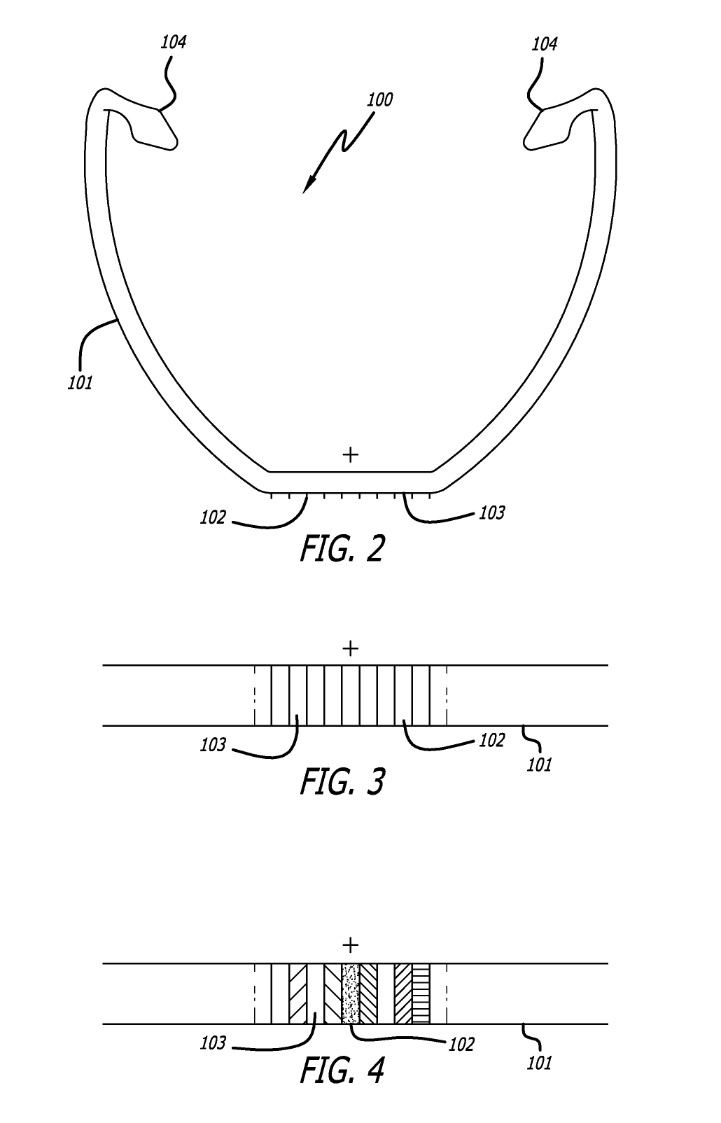

FIG. 2 depicts a side view of an exemplary plagiocephaly detection device 100. A plagiocephaly detection device comprises a one piece structure 100 having curved calipers 101, planar connector 102 and ear tips 104. The ear tips are positioned at distal ends of the curved calipers 101. The planar connector 102 bridges the proximal ends of the curved calipers. The calipers 101 may be approximately 4″ apart from the distal ends and approximately 3″ apart from the ear tips. The height of the device (distance vertically between ear tips and the planar connector) may be approximately 3″. The device may be approximately 1\4″ deep and 1\8″ thick all around. The curved calipers 101 of the device provide flexibility and comfort to infants as its curved calipers complement the curvature of the infants face. The planar connector 102 comprises indicia 103, or spatial markings, which fall under the infant's nose while the caliper's ear tips 104 are positioned in the infant's ears. The device 100 is flexible and hence by holding the calipers 101, one can easily push in or pull away in lateral direction. This allows flexibility on placing the device on the infant.

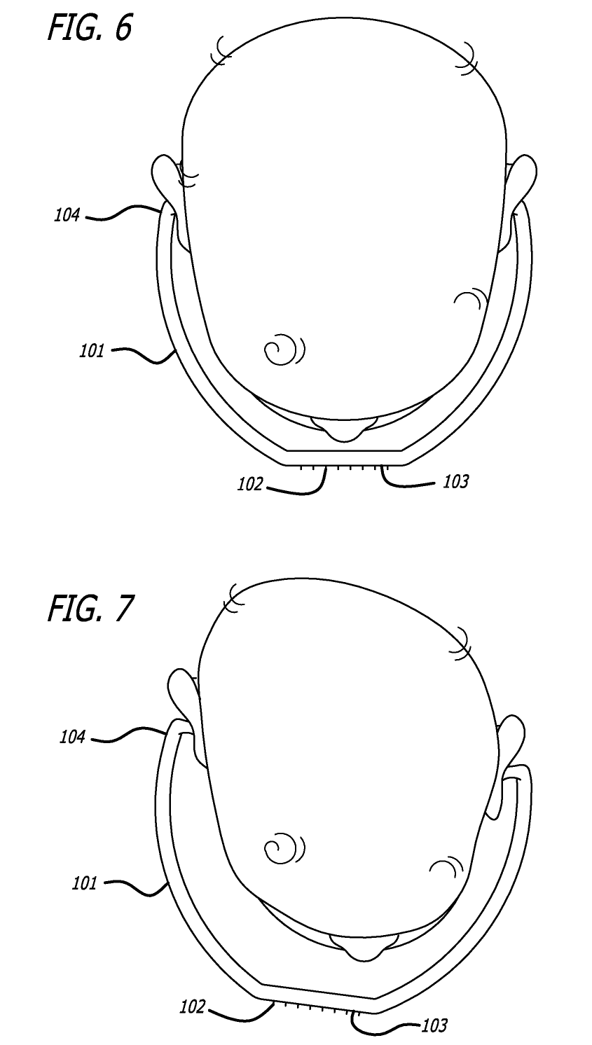

FIGS. 3 and 4 depict front and oblique views of the device showing the indicia 103 of the connector 102. In the exemplary embodiment the indicia 103 are molded onto the planar connector 102 as seen in FIGS. 2 and 6 wherein the indicia 103 in the form of grooves are depicted. In an alternate embodiment, the indicia 103 are prepared on a plastic film-like material and glued onto the connector 102, such as a sticker, as seen in FIG. 4. The + sign in FIGS. 3 and 4 represents the center marker position of the indicia 103.

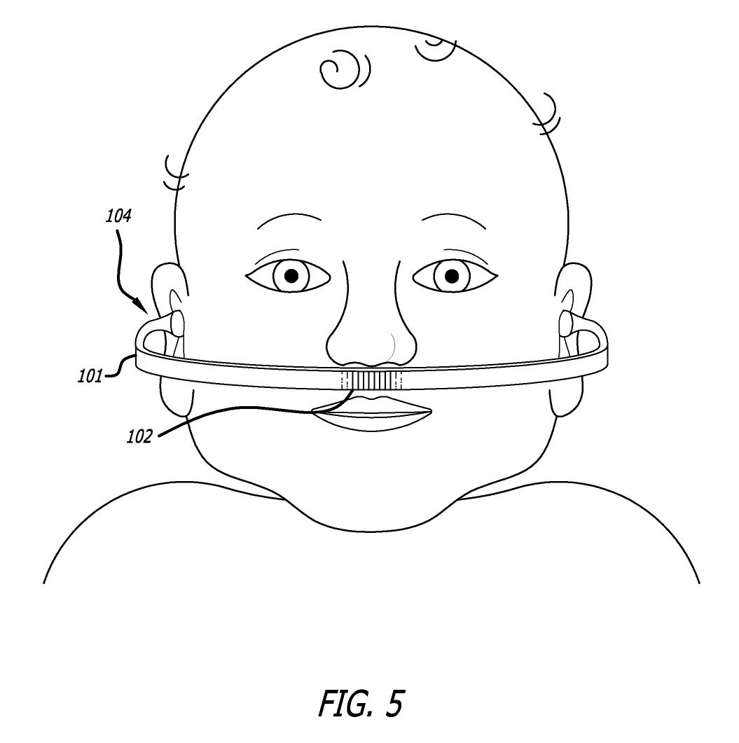

The device can be placed on the infant with ear tips attached to both ears and the planar connector 102 placed under the infant's nose. The connector 102 falls between the newborns nose and upper lip as shown in FIGS. 5 and 6. The markers 103 provide visual indication of plagiocephaly to the caregiver. In the absence of occipital plagiocephaly, the midline of the infant's nose aligns with the middle position marker of the indicator indicia 103 of the connector 102, as displayed in FIGS. 5 and 6. When plagiocephaly exists, the nose's midline does not align with the center marker (FIG. 7). The markers may also serve as an indicator of severity of plagiocephaly, depending on the measured distance of misalignment from the center. The lateral position markers may be equal distance from each other. In one embodiment, the markers may be one millimeter apart. In another embodiment, the markers may comprise colored regions to show relative change in position.

FIG. 7 represents use of the device on an infant. As depicted, the connector's center position marker is severely offset from midline of the infant's nose. The right side of the skull, along with the right ear, is shifted forward. The center position marker is not in alignment with the nose. The semi-rigid construction of the device 100 and its material makes it light, flexible, durable and capable of accurate detection of plagiocephaly. The device also does not require any calibration, adjustment or special skills to use, and therefore makes it easy and convenient to use. Some embodiments of the device may be configured to fit newborns of six months or younger, while others may be configured to fit infants up to nice months old.

The previous description of the disclosed embodiments is provided to enable any person skilled in the art to make or use the plagiocephaly detection devices and methods. Various modifications to these embodiments will be readily apparent to those skilled in the art, and the generic principles defined herein may be applied to other embodiments without departing from the spirit or scope of the plagiocephaly device and methods. Thus, the plagiocephaly detection devices and methods are not intended to be limited to the embodiments shown herein but are to be accorded the widest scope consistent with the principles and novel features disclosed herein.

1. A semi-rigid device for detecting plagiocephaly in an infant comprising:
a) a pair of calipers each having a curved section and having a proximal and a distal end, wherein each distal end has an ear-tip configured to rest inside of the infant's ear; and
b) a connector segment located between the calipers that connects the proximal ends of the calipers, wherein the connector segment has a planar outer surface comprising a central position marker located at the center of the connector segment.

2. The device of claim 1, wherein the planar outer surface of the connector segment further comprises lateral position markers aligned an equal distance from each other, on opposite sides of the central position marker, and positioned along a linear axis that originates at the center of the planar outer surface of the connector segment and extends outwardly toward the proximal ends of the calipers.

3. The device of claim 2, wherein the central position marker and lateral position markers are affixed to the planar outer surface of the connector segment.

4. The device of claim 2, wherein the central position marker and lateral position markers are integrated into the planar outer surface of the connector segment.

5. The device of claim 1, wherein the calipers and connector segment comprise plastic.

6. The device of claim 1, wherein the ear-tips point toward the connector segment.

7. The device of claim 1, wherein the device is configured so that each of the ear-tips can simultaneously rest inside of each of the infant's ears.

8. A method of detecting plagiocephaly in an infant with a caliper comprising:
a) positioning a semi-rigid caliper on the infant, wherein the caliper comprises:
i) a pair of calipers each having a curved section and having a proximal and a distal end, wherein each distal end has an ear-tip configured to rest inside of the infant's ear; and
ii) a connector segment located between the calipers that connects the proximal ends of the calipers, wherein the connector segment has a planar outer surface comprising a central position marker located at the center of the connector segment; and
b) determining the spatial relationship between the infant's nose and the central position marker.

9. The method of claim 8, wherein the distal ends of the calipers are simultaneously placed into the infant's ears.

10. The method of claim 8, wherein the caliper is configured to be placed on infants up to nine months old.

11. The method of claim 8, further comprising determining the degree of misalignment between the central position marker and the infant's nose.
